(19) United States
(12) Patent Application Publication
(10) Pub. No.: US 2010/0185279 A1
(43) Pub. Date: Jul. 22, 2010

(54) SURGICAL FASTENING DEVICE

(76) Inventor: Sujay Shad, New Delhi (IN)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/376,518

(22) PCT Filed: Jul. 31, 2007
(86) PCT No.: PCT/GB2007/002860

§ 371 Date: Aug. 3, 2009

(30) Foreign Application Priority Data
Aug. 14, 2006 (IN) 815/KOL/2006
Sep. 26, 2006 (GB) 0618903.9

(51) Int. Cl. A61F 2/24

(52) U.S. Cl. 623/2.38

(57) ABSTRACT

Apparatus for the affixing of prosthetic, mechanical devices to body tissue. Often referred to as a “surgical attachment ring.” At least a portion of the outside edge of said ring is substantially of a non-convex profile. The application may also characterised by the feature of pre-attached or pre-loaded sutures or by the presence of one or more suture—receiving passageways in the body of the ring.

### FIELD OF THE INVENTION

This invention concerns apparatus for the affixing of prosthetic, mechanical devices to body tissue. It has particular application to the affixing of such devices to openings such as stoma and of the attachment of replacement valves to vascular tissue and even more specifically to human hearts, and especially concerns the affixing of prosthetic aortic and mitral heart valves and mitral and tricuspid annuloplasty rings.

### BACKGROUND AND PRIOR ART KNOWN TO THE APPLICANT

Neither prosthetic, mechanical valves nor the means of attachment of such valves to organic tissue, including that of man, are new. Prosthetic, mechanical valves uniformly have a sewing ring (also known as a “collar”) attached to the valve housing before surgery. The sewing ring is commonly made of a woven material, typically of a polymer of some sort. It is attached to the outside of the housing of the valve, either directly, with an adhesive substance, or via a built in metal ring, which may likewise be bonded with an adhesive material. A common configuration of the positions of the valve and the sewing collar when attached are illustrated in FIG. 1. In this figure, 1 indicates the valve generally, and 2 the sewing ring generally. 3 shows the point of bonding between the valve and the ring, while 4 serves to emphasise the hollowness of the ring. The space at 4 is important because this is the functional space through which a given substance, for example blood, may pass. 5 shows the functional area of the valve. This functional area may further be reflexively defined as the effective orifice area of the valve left exposed by space 4. The purpose of the sewing ring is to enable surgeons, or other personnel fulfilling a similar function, to pass sutures longitudinally through the sewing ring, and similarly and sequentially through an abutting tissue wall in order that the ring, and therefore also the valve, may properly be attached (or more commonly, “sewn”) to the necessary area of the patient's body.

A range of heart valve attachment mechanisms are known, for example:

U.S. Pat. No. 6,176,877 describes a two-piece prosthetic heart valve, having a fabric sewing for attachment to cardiac tissue.

UK Patent GB 1,093,599 describes an artifical cardiac valve, having a coarse mesh (i.e. porous) synthetic fibre covering.

U.S. Pat. No. 6,0936,074 describes a multi-part heart valve assembly, two components of which are of permeable felt.

U.S. Pat. No. 3,996,623 describes a method of implanting a prosthetic device using an annular sewing ring whose outer surface is an interlaced or knitted fabric.

U.S. Pat. No. 6,287,339 describes a heart valve prosthesis attachable by a cloth sewing cuff.

It is usually the case that all but elderly patients are delayed from undergoing heart valve replacement surgery until later in life. This is because heart valve disease is not an immediately life-threatening condition, and also because of the shortcomings of both prosthetic and tissue valves, shortcomings which are exacerbated by the current design of the sewing ring. The symptoms of a given heart valve disorder will thus be managed with medication for as long as possible. Such a course of medication may in itself have a considerable number of negative side effects for the patient. In addition, the cost of the drugs used in the course, as well as all the attendant costs related to the management of a patient with a chronic condition, create a long term cost burden to the healthcare system. The combination of the condition itself, the course of drugs, and the social and psychological experience of being a chronic heart patient will often negatively restrict the patient's quality of life. Moreover, the continuing weakness in the integrity of a given patient's heart may serve to exacerbate the likelihood of developing secondary cardiac conditions, as well as the subsequent speed of their development and seriousness, if caught. Improving the design of the sewing ring would serve to ameliorate some of the problem conditions associated with mechanical, prosthetic heart valve installation and functionality and therefore also some of the factors which currently go some way to determine the decision—making parameters surrounding valve replacement operations. The negative factors associated with sewing rings are discussed in detail below.

Often, the sewing ring can be a major impediment to important valve functions, and thus to the health of the patient as a whole. First, it may increase the seriousness of problems intrinsic to the fitting of mechanical, prosthetic heart valves. Second, it may also increase the likelihood of a given patient suffering from conditions associated with the operation. Third, the design of the ring brings problems and attendant risks with it. Fourth, the design of the ring governs the nature of the attachment process, and this brings problems and attendant risks. Six of the key problems may be identified:

The first problem is that the sewing ring occupies some of the space which would better be left for the functional part of the valve. Large valves have rings that protrude at least 4 mm from the circumference of the tissue wall, and the subsequent reduction of the valve area is significant, since blood has to be pushed through a smaller aperture increasing pressure. This results in the heart having to work harder, generally causing heart muscle to thicken—a condition known as “left ventricle hypertrophy”. Physiologically, this is akin to narrowing of the aortic valve or stenosis. This “functional stenosis” through a smaller than possible effective orifice area is believed to be the reason that after aortic valve replacement in particular, there is inadequate regression of left ventricular hypertrophy. Various design modifications have been unsuccessfully produced by the industry to offer a solution to this problem which is now well known in cardiac surgical literature as patient-prosthesis mismatch.

The second problem caused by with current sewing ring design is that in all known manifestations of the ring, it is convex on the side of its interaction with the patient tissues. The process of attaching a sewing ring to the inside of a patient's heart generally involves making a ring of stitches with suture. At any given longitudinal point in the ring, the suture passes in and out of the tissue only once. This means that when the suture is made taut, in order that a best fit may be achieved between the valve and the tissue wall, there is only one longitudinal point at which force bringing tissue and valve together is exerted. This process therefore forces the tissue to develop into a convex shape as well. Since the interaction of the tissue and the sewing ring is convex to convex, the surface coaptation will be of a relatively small area. There may even be small spaces in between the sewing collar and the tissue where perivalvular leakage could occur. Even in the absence of such immediate leaks, the aforementioned small surface area will leave the join prone to developing leaks.

The third problem is that the sewing ring, all its present forms, is characterised by the woven quality of the ring itself. This woven form imbues the ring with the undesirable quality of porousness. The ring itself can therefore harbour infectious agents. Upon installation in the cardiac region, the sewing ring may provide a seat of infection and indeed it often does, causing of 2% early and −2% late infective prosthetic valve endocarditis. Such porousness may also facilitate a degree of perivalvular leakage. It is certain though that the interstices of a fabric do provide residence to bacteria to reside and multiply. This bacterial infection or fungal infection was also addressed by the industry by creating a silver impregnated sewing ring, however the rings were rejected by the body rather rapidly. Removing the sewing ring completely would reduce the potential for infection build up and will possibly completely eliminate it.

The fourth problem is that of Stenosis—the process whereby valve apertures are narrowed by the build up of proteins on the tissue walls, a narrowing which reduces the effectiveness of the valve. This is called pannus in cardiac surgery literature. Stenosis is thought to be caused by an immunological reaction to the presence of foreign materials in the body. The reaction takes the form of a build-up of protein around a given object. The reaction is a method utilised by the body to isolate the foreign object from the rest of the body, and in particular the circulatory system. As well as reducing the efficacy of the blood vessel, such a build-up increases the chance of thrombosis where blood clotting actually occurs. It is widely held that the larger the quantity of foreign material at a given site, the greater the occasion for and occurrence of protein build-up. Given that the sewing collar forms a significant part of the foreign matter present subsequent to the fixing of a mechanical prosthetic valve into a patient's heart, and moreover the texture of the sewing collar being fibrous forms an even more significant part of the surface area of the foreign matter present in the bloodstream and moreover the sewing collar makes up the totality of foreign matter actually in contact with the cardiac walls, it is desirable that the sewing collar or related device, be of the smallest size possible.

The fifth problem is that prosthetic material promotes thrombosis. Therefore the key result is that a prosthetic valve recipient requires a permanent, anticoagulant medication which is regularly monitored (to inform the dosage level required) to help prevent thrombosis. The anticoagulants themselves tend to cause serious morbidity and even death in up to 2% of patients annually. Altering the design of the collar will help sever this concatenation of problems.

The sixth problem is that it is possible that the sewing ring itself promotes thrombogenicity (the formation of blood clots). To counteract this side-effect it is thought that there is a need for a higher degree of anticoagulation, which aside from exacerbating any perivalvular leakage, may also affect the patient in myriad other ways.

In summary, the shortcomings of the sewing ring as an aspect of mechanical, prosthetic heart valve replacement make a significant contribution to the physical drawbacks and therefore also to the body of empirical knowledge drawn on during the pre-operative decision-making process, the result of which is that many patients who need a replacement valve do not receive it until late in life. Improving the sewing collar or replacing it with a different device which performed the same positive functions would improve heart valve functionality and would therefore enable all patients to receive earlier intervention and a lower medication, resulting in improved quality of life and longer life expectancy.

It is an object of the present invention to attempt a solution to these and other problems.

### SUMMARY OF INVENTION

In the first broad independent aspect, the invention presents a surgical attachment ring. The invention is characterised by the feature that at least a portion of the outside face of said ring is substantially of a non-convex profile.

The ring has two opposing faces, which will be referred to as an outer face and an inner face, so arranged that the outer face runs around the inner face, the inner face defining a passageway, and being arranged in such a way that it runs through 360 degrees. The outer face also runs through 360 degrees. The inner and outer faces are separated by the substance, or body, of the ring. The two surfaces that join the faces of the ring will be referred to as the ends of the ring.

A surgical attachment ring is designed to do the same principal task as the sewing rings which make up the prior art. Viz.: the aiding of the attachment to or implanting into a given area of biological tissue a given object, during surgery, such as a replacement heart valve.

Shaping the profile of the ring in such a way that it is non-convex is particularly advantageous because it allows for the abutment of the ring and the tissue to which it is to be attached to be as large as possible, and this good fit greatly enhances the integrity of the coaption, which in turn will reduce the occurrence of perivalvular leakage around the suturing.

A further advantage is that the close fit of the contact will lessen the chances of infection occurring between the surface of the ring and the tissue wall.

A further advantage is that, in the case of a match between a convex tissue profile and a concave ring profile, the ring will fit naturally to a degree, which will be advantageous in the initial surgical suturing process.

Such a profile would also be advantageous in holding the ring and valve in place should one or more of the sutures fail.

In a subsidiary aspect of the present invention the entire outer face of said ring is substantially of a non-convex profile.

Such an arrangement is particularly advantageous inasmuch as the regularity of the structure promotes strength and integrity within the structure.

The regularity of the structure is further advantageous in that it allows for an extra degree of uniformity during any surgical attachment procedure pertaining to it.

In a further subsidiary aspect of the invention, the surgical attachment ring further comprises guides attached to or forming part of the ring in such a way as to guide, in use, a suture.

Having such guides for the sutures, which may in a given embodiment take the form of cleats, would be advantageous in that they would facilitate quicker, neater operations.

Such guides may also be of particular advantage in “transcatheter sugery”, and other, related, minimally invasive surgical techniques, since the presence of guides would allow for regularity and uniformity to be introduced to the suturing process, an advantage over the irregularities in suturing necessitated by a woven collar.

In a further, subsidiary embodiment of the invention, the surgical attachment ring is characterised by the provision of a plurality of suture—receiving passages passing through the body of said ring and so circumferentially spaced as to be able to attach the ring, in use, and with sutures drawn in place, adequately to the tissue surrounding the ring.

By incorporating holes in the ring (that may form part of the supporting structure of, for example, a heart valve), to enable direct attachment of the ring to body tissues, the requirement for an additional fabric—like sewing ring is obviated. In this way, the functional size of the passageway through the valve may be increased.

The presence of passages would provide further advantage during surgery, since, the number of sutures required and the positioning of those sutures would, to some extent have been predetermined. This opportunity to predetermine may be used by the designers, manufacturers or other such interested parties to ensure the optimal performance of the ring.

Particular arrangements of passages are advantageous in that they would promote regularity of suturing. This, again, would reduce the risk of perivalular leakage. Some of these are mentioned below, and all are advantageous in that they promote an increasingly regular and secure suturing technique.

In a further subsidiary embodiment of the invention, the surgical attachment ring has the passages mentioned above, but at least one of said suture-receiving passages extends between opposite ends of said ring.

In a further subsidiary embodiment of the invention, the surgical attachment ring has at least one of said subsidiary passages extending between an end and the outside face of the ring.

In a further, subsidiary embodiment of the invention, the surgical attachment ring comprises at least one pair of passages, one member of a given pair extending between a first end of the ring and the outer face of the ring and the other member of the given pair extending between the corresponding other end of the ring and the outer face of the ring and in cases of more than one pair, all passages extending between a first end of the ring and the outer face of the ring extend to substantially the same radial plane of the ring on the outside face, while the other member of the given pair extending between the corresponding other end of the ring and the outer face of the ring extend to substantially the same radial plane of the ring on the outside face, although the first and second radial planes mentioned herein may not necessarily be the same.

In a further, subsidiary embodiment of the invention, the surgical attachment ring further comprises guides located proximate to said passages, configured in use to guide a suture passing through said passages.

The advantages of guides have already been stated above. It may be added that when used in tandem with a given passage configuration, sutures will be more controllable during surgery, making such procedures even quicker, safer and cheaper.

In a further, subsidiary embodiment of the invention, the surgical attachment ring further comprises at least one suture located within a passage.

In a further, subsidiary embodiment of the invention, the surgical attachment ring further comprises at least one suture attached to the ring.

In a further, subsidiary embodiment of the invention, the surgical attachment ring further comprises a multiplicity of sutures attached to said ring.

The advantages of the sutures mentioned in the previous three paragraphs, which may be referred to in the case of the former as “pre-loaded”, and in the case of the latter as “pre-attached” are manifold, whether one or both types are used. Clearly, if a suture is pre-loaded or pre-attached, one does not need to introduce it separately in a given surgical operation. There is one less component to the operation and one less task for the surgical team to perform.

Second, since the sutures would be accurately pre-loaded or pre-attached on the valve it would increase the accuracy of the suturing, and reduce the chances that stitching and/or placement would be poor.

The use of pre-loaded or pre-attached sutures may also be advantageous in that it excises the need to pass the suture through the collar in the manner which woven collars require. This saves not only time, but also reduces the volume of space required to perform the operation in that the movement required to push the suture through the collar is no longer required.

By excising the movement discussed in the above paragraph, the attendant likelihood of damaging surrounding tissue walls during said movement is also reduced to nil.

Moreover, and particularly in mitral valve replacement, the surgeon often secures the valve with sutures that are super- of sub-valvular only, dependent on access and availability of the remaining annulus on excision of the damaged valve. Standardisation of the suturing via a pre-loaded pre-attached element would be of particular advantage in such situations, provide an increased opportunity for efficient and comprehensive attachment of the implant.

In combination, the changed shape of the device and the introduction of pre-loaded and/or pre-attached sutures allow for envisaged embodiments of the device to be far smaller than existing surgical attachment rings. The reduction in the size of the ring will increase the functional surface area of the valve, improved haemodynamic function, which in itself will result in less turbulent blood flow and decrease the propensity for the laying down of proteins around the implant, thrombosis and improved regression of left ventricular hypertrophy.

The new features will also allow for the ring to be manufactured without the woven quality which has characterised such rings previously. The woven texture of these rings has been identified both as a seat of infection and as an exacerbating factor in cases of Stenosis. By removing the woven quality, the likelihood of either occurring is greatly reduced.

The attachment ring maybe supplied with a “rescue pack”, comprising at least one needle which has the correct properties to allow, in use, for the efficient manoeuvring of sutures into, out of and through the suture—receiving passages of the ring. This could be used in the event that one of the pre-loaded or preattached sutures breaks. In a preferred embodiment, such a pack would comprise crochet-type fine needles. A slightly curved crochet would be required to firstly pass a fresh suture through the patient's tissues and a second straight crochet type needle would be required to retrieve the suture through the valve attachment ring.

In a particularly preferred aspect of the invention, there is provided a surgical attachment ring composed of a substantially non-porous solid substance or substances and whose peripheral and radially outside tissue-contacting face either comprises a continuously curved non-convex portion or is flat across substantially its entire surface.

Preferably, the entire outside face of the ring has a substantially non-convex profile.

In an alternative preferred embodiment of this aspect, a portion, only, of the outside face of the ring has a non-convex profile.

In any aspect of the invention, it is preferred that the outside face of the ring is symmetrical about its mid-circumferential plane.

Also in any aspect of the invention, it is particularly preferred that the outside face of the ring has an L-shaped profile.

Also in any aspect of the invention, it is particularly preferred that the outside face of the ring incorporates a number of suture cleats. More preferably, the outside face of the ring comprises a number of raised ribs through which run suture-receiving passages.

Also included within the scope of the invention is a surgical attachment ring substantially as described herein with reference to and as illustrated by any appropriate combination of FIGS. 2 to 6 of the accompanying drawings.

Also included within the scope of the invention is a prosthetic valve incorporating a surgical attachment ring of the type described herein. Preferably, the valve is a prosthetic heart valve.

### BRIEF DESCRIPTION OF THE DRAWINGS

The practical embodiments of the invention will now be described with reference to the accompanying drawings in which:

FIG. 2 shows a surgical attachment ring embodying the invention in schematic view.

FIG. 3 shows a portion of the surgical attachment ring viewed in schematic view.

FIG. 4 shows both a plan and a cross-sectional view embodying the invention with the cross-section directly below the diagram embodying a cross section at the point indicated by the arrows marked A

FIG. 5 shows both a plan and a cross-sectional view embodying the invention with the cross-section directly below the diagram embodying a cross section at the point indicated by the arrows marked B.

In FIG. 6 a further embodiment of the invention may be seen in schematic cross section.

FIGS. 7, 8, and 9 show different embodiments of the guide feature within a schematic view of a section of the ring.

FIG. 10 shows the 2 fine needles used in the rescue pack.

### DETAILED DESCRIPTION OF THE DRAWINGS

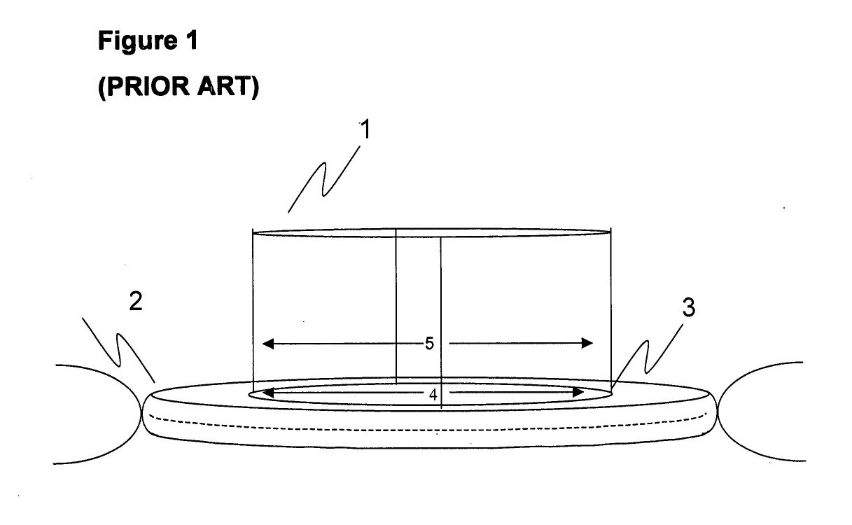

The surgical attachment ring in FIG. 2 is intended to provide a means by which a prosthetic surgical valve, in particular a surgical heart valve, or other such prosthetic device may be attached to organic tissue or a stoma. The surgical attachment ring is generally denoted by 6. It is composed of solid, non-porous substance or substances. This is in order to avoid the absorption by the surgical attachment ring of the fluid which passes through the attached valve or stoma. In so avoiding the absorption of said fluid complications which may arise such as, in the case of a heart valve surgical attachment ring, stenosis, or in surgical valve attachment more generally, rejection of the surgical attachment ring by the given surrounding tissue are also avoided. The ring 6 abuts the annulus of surrounding tissue 7. The abutment 8 is between a concave collar and convex tissue surrounds. In a different embodiment it may be that the collar has a flat edge. In any event, this attachment will not be convex to convex. In any embodiment the ring will have an passageway 9 through which fluid will pass. The composition and construction of the surgical attachment collar will allow the surgical attachment collar to be manufactured at a given diameter 10 while allowing the functional diameter of the valve mechanism 11 to be maximised. The inventor notes, in relation to heart valves, that by decreasing the radial area occupied by the surgical attachment ring in relation to the surrounding tissue, the valve's functional area could occupy up to 90% of the aperture.

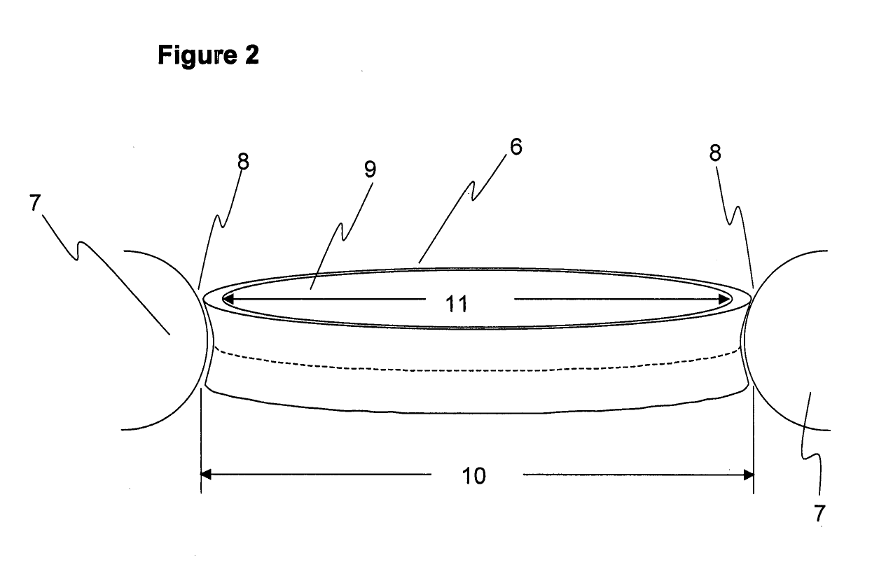

FIG. 3a shows a portion of the surgical attachment ring in a perspective view. The portion of the surgical attachment ring is shown generally at 12. The outer face of the ring 13 is, during surgical procedure, brought into contact with the tissue walls. The outer face is defined by the ends 14, 29 of the ring, or which in this diagram only one 14 is visible. The ring has a number of suture—receiving passages 15 which run through it. A suture receiving passage may run from either end of the ring to the outer face, forming openings 16, 17/18, 19.

20 denotes a suture. This suture may be preloaded, in a number of ways. One such embodiment of a pre loaded suture would be that it travelled through a suture receiving passage, running from one end 14 to the outer face 13 via openings 21, 22 and then travelled through a second adjacent suture receiving passage, running from the outer face 13 to the second end 29, via openings 23, 24. This embodiment features a number of guides 25, around the circumference of the ring adjacent each pair of suture receiving passages, forming in this embodiment a unit with pairs of suture receiving passages, by which it is meant passages in a similar spatial and/or operative relationship to 21-24. In a preferred embodiment this guide may be a cleat. In a further preferred embodiment this guide may be an indentation in the surgical attachment ring. In this embodiment the suture has a head 26, which may for example be made of metal or a polymer and which in this embodiment is made from a different material to the length of the suture 27.

FIG. 3b is a cross-section of FIG. 3a, in a vertical (as illustrated) plane through a pair of suture receiving passages. The suture-receiving passages may take various paths within the body of the ring, 16, 17.

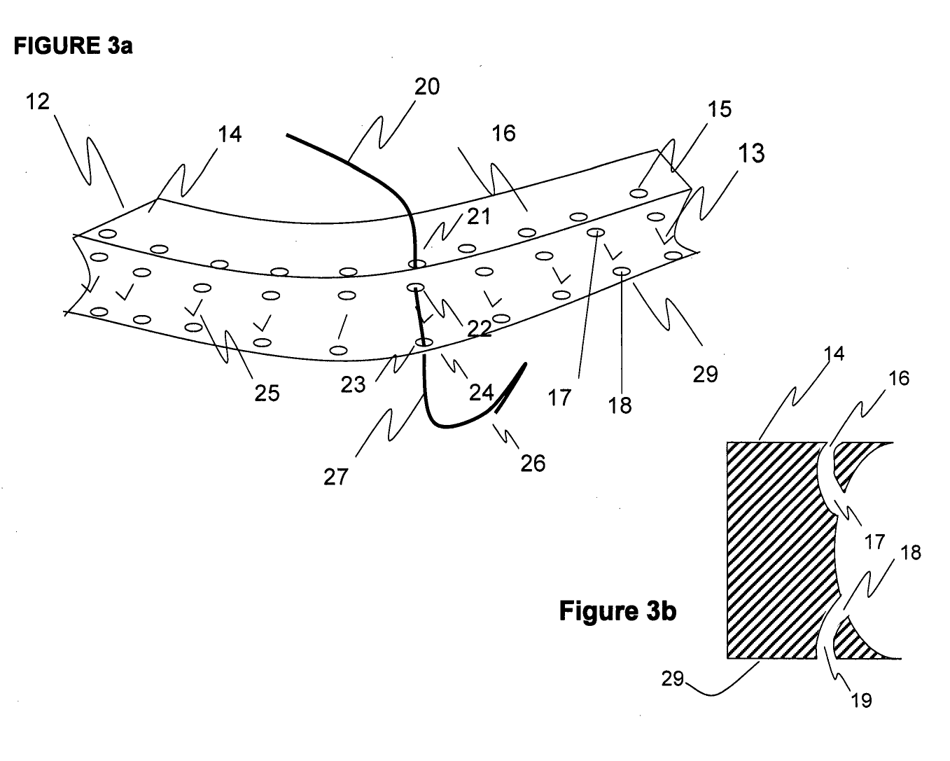

FIG. 4a is a plan view of an embodiment of the surgical attachment ring 28, herein embodied in a generally quadrilateral shape. The embodiment has a passageway 9 through which a given fluid will flow. This embodiment features a number of suture-receiving passages 30 with visible openings on the end 31. It is noted that there may be any number of passages in the ring provided they are sufficient to perform the function of fixing the given surgical attachment ring to the valve or stoma. They may also be spaced in a variety of different ways, in terms not only of the distance between given passages but also in terms of their proximity to the passageway 9 through which fluid will flow, which is formed by the shape of the inner face 32.

FIG. 4b is a cross-section of FIG. 4a at A; the passage 9, is defined by the inner face 32. The ring's body or substance is shown in cross—section 33, 34. Given suture-receiving passages 35, 36, 37 and 38 may take different paths within the body of the ring.

FIG------------------------------------------------------------. 5a is a plan view of a further embodiment of the ring 39. The ring is of a generally circular shape 40 with suture-receiving passages in the edge of the ring 41. The ring has a passageway, 42 defined by the inner face 43.

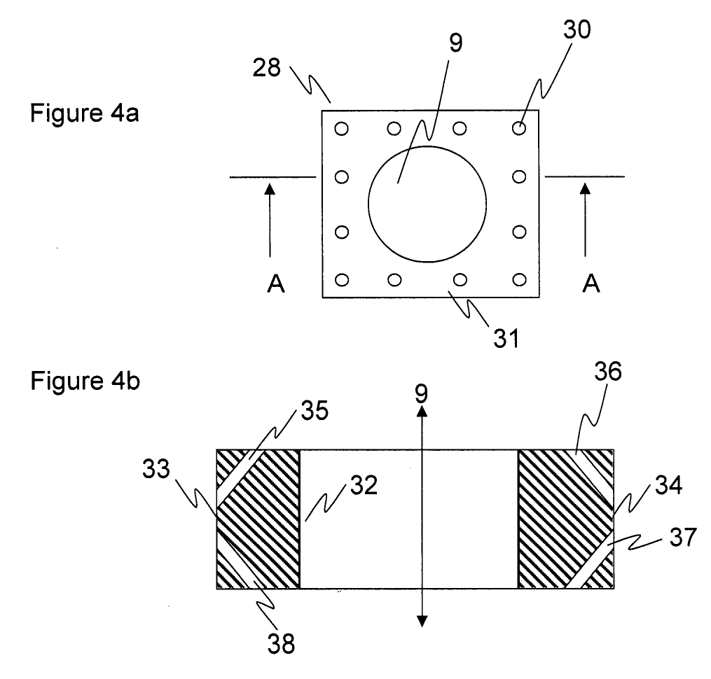

FIG. 5b is a cross-section of 5a at B, showing a passageway 42 defined by the inner face 43. In the diagram, you can see cross-sections of the body or substance of the ring 44. The suture-receiving passages may take a variety of paths through the body of the ring, of which some are illustrated: 45,46,47,48.

FIG. 6 shows a further embodiment of the ring in schematic cross-section. The fluid receiving passageway 63 is defined by the inner face of the ring 49. This cross-sectional embodiment allows viewing of a cross-section 50, 51 of the surface of the ring. In this embodiment, the cross-sectional profile allows viewing of trenches in the ends of the ring 52, 53. Note also that such trenches or indentations need not be circular and can take many forms and be many in number. The suture-receiving passages may take a variety of paths through the body of the ring, of which some are illustrated: 56, 57, 58 and 59. A suture is shown 60. This could either be a pre-loaded or a non-preloaded, i.e. manually loaded, suture. The path of the suture through the passage is shown in the cross-section 61. Suture 62 may also be attached to the ring itself. The point of attachment between the suture and the ring 64 may take place upon any portion of the ring. The methods and means of attachment may be any deemed suitable by individuals skilled in the art.

FIG. 11 illustrates yet a further embodiment of a surgical attachment ring 39 according to the invention, illustrated in cross-section (FIG. 11a) and plan (FIG. 11b). In this embodiment, the ring 39 has an outer face 71 having an “L-shaped” profile, creating a concave surface that may be mated, in use, against a patient's tissue. The top (as illustrated) end of the ring 14 is provided with a plurality of suture-receiving passages 56, 57, through which sutures may be passed., As described above, the suture-receiving passages may also be pre-loaded with sutures. A particular advantage of this single-lipped embodiment of the attachment ring, is that is easier for the surgeon, in some circumstances, to insert and position it in correct alignment with the patient's tissues. Also, as only a single ring of suture-receiving passages 56, 57, is provided (i.e. all passages are located on the same face of the ring) access through the central passageway 42 of the ring is not required when suturing the ring in place.

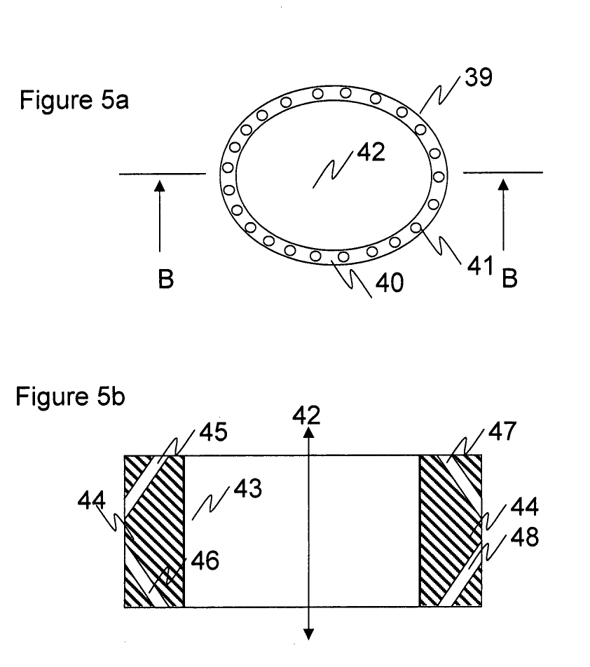

FIG. 7 is a schematic embodiment of an outer face of the ring. The ring is generally indicated at 70. The outer face of the ring 71 is concave. It features a guide comprising raised portions 72, 73 protruding from the surface of the ring between which in use a suture 74 may be guided on a particular path.

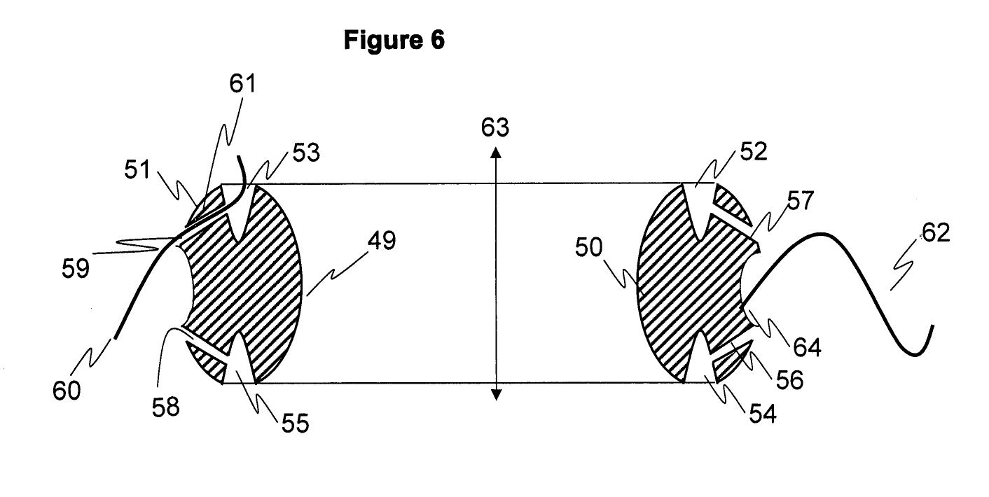

FIG. 8 shows a schematic embodiment of an alternative outer face of a ring. The ring is generally indicated at 80. The outer facer of the ring 81 features a guide in the shape of a cleat. In this embodiment the cleat comprises a shaft 82 which terminates in a boss 83. In use a suture 84 may be wrapped around the cleats fastening and guiding it in use.

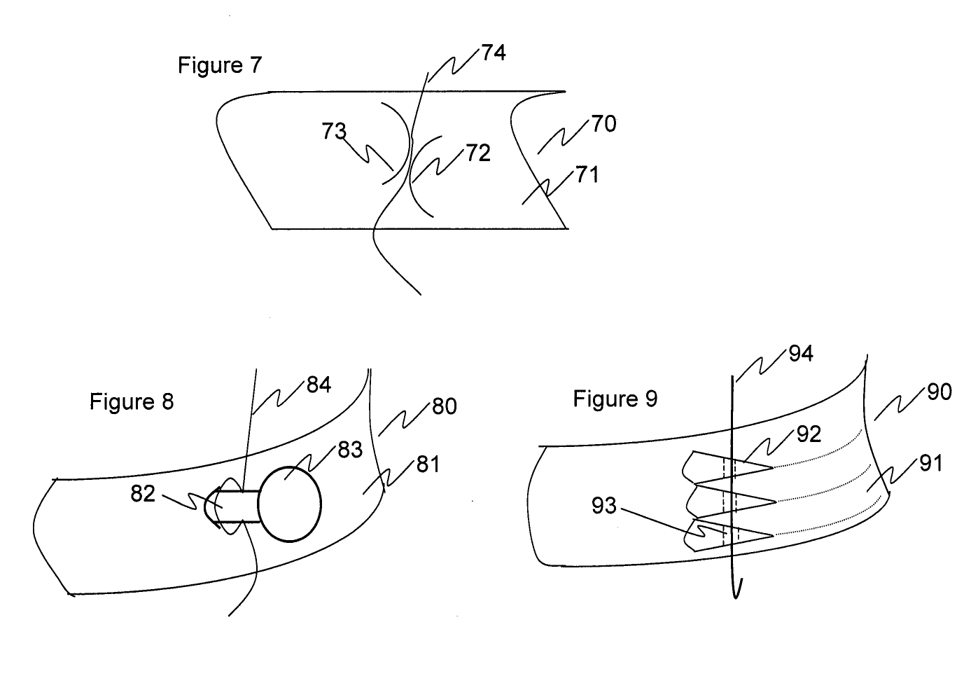

FIG. 9 shows a further schematic embodiment of an alternative outer face of the ring. The ring is generally indicated at 90. The convex outer face of the ring 91 features a guiding system comprising a number of raised ribs 92, here shown in cross-section, through which run suture-receiving passages 93 through which in use a suture 94 may be guided so as to achieve a sureness of fit.

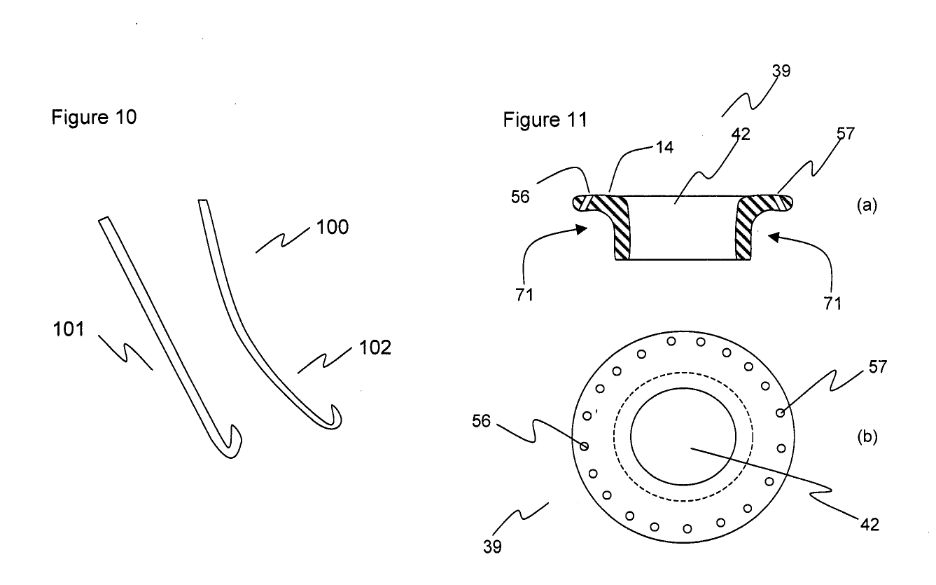

FIG. 10 illustrates the surgical rescue kit. The rescue kit is indicated generally at 100. The rescue kit consists of two flexible suturing implements 101, 102, having a crotchet hook on one end, and which in use are of dimensions sufficient to allow them to be used in the removal of broken sutures from passages in the ring and also to allow their use in passing sutures through said passages.

1. A surgical attachment ring composed of a substantially non-porous solid substance or substances and whose peripheral and radially outside tissue-contacting face either comprises a continuously curved non-convex portion or is flat across substantially its entire surface.

2. A ring according to claim 1 and in which the entire outside face of the ring has a substantially non-convex profile.

3. A ring according to claim 1 and in which a portion, only, of the outside face of the ring has a non-convex profile.

4. A ring according to claim 1 and in which the outside face of the ring is symmetrical about its mid-circumferential plane.

5. A ring according to claim 1 and in which the outside face of the ring has an L-shaped profile.

6. A ring according to claim 1 and in which the outside face of the ring incorporates a number of suture cleats.

7. A ring according to claim 6 and in which the outside face of the ring comprises a number of raised ribs through which run suture-receiving passages.

8. A surgical attachment ring substantially as described herein with reference to and as illustrated by any appropriate combination of FIGS. 2 to 6 of the accompanying drawings.

9. A prosthetic valve incorporating a surgical attachment ring in accordance with claim 1.

10. A prosthetic heart valve incorporating a surgical attachment ring in accordance with claim 1.
